(19) United States
(12) Patent Application Publication
(10) Pub. No.: US 2024/0033513 A1
(43) Pub. Date: Feb. 1, 2024

(54) A MUSCLE STIMULATION AND MONITORING APPARATUS

(71) Applicant: IMPERIAL COLLEGE INNOVATIONS LIMITED, London (GB)

(72) Inventors: Ravi Vaidyanathan, London (GB); Samuel Wilson, London (GB); Dario Farina, London (GB); Thomas Watts, London (GB)

(21) Appl. No.: 18/267,907

(22) PCT Filed: Dec. 16, 2020
(86) PCT No.: PCT/GB2021/053339

§ 371 Date: Jun. 16, 2023

(30) Foreign Application Priority Data
Dec. 16, 2020 (GB) 2019867.7

(51) Int. Cl. A61N 1/36; A61N 1/02

(52) U.S. Cl. CPC A61N 1/36031; A61N 1/36003; A61N 1/36034; A61N 1/025

(57) ABSTRACT

An apparatus comprising: at least one processor; and at least one memory including computer program code, the at least one memory and computer program code configured to, with the at least one processor, cause the apparatus to: receive a sensor output from a mechanomyography sensor configured to monitor muscle activity of a muscle in a human or animal body; and control, in response to the received sensor output, an electrical stimulus applied by a muscle stimulator to the muscle to modify said muscle activity, wherein the electrical stimulus is applied with an amplitude below the motor threshold of the muscle simultaneously during monitoring of the muscle activity by the mechanomyography sensor.

### TECHNICAL FIELD

The present disclosure relates to the monitoring of muscle activity in human or animal bodies and, in particular, concerns an apparatus and associated methods for controlling an applied muscle stimulation in response to the monitored muscle activity.

### BACKGROUND

Physiological symptoms of neural degeneration or damage of the brain such as tremor, slow movement and muscle rigidity occur when the communication between the brain and the muscles is partly interrupted or degenerated. This type of impairment can be mitigated by changing the sensory input (that is, the sensation signals) to the brain, leading to a decrease in presentation of the symptoms. Additionally, extended use of such symptom suppression over time can prompt neurological changes within the brain and provide a lasting therapeutic effect. The latter mechanism is based on the neuroscientific basis of brain plasticity, according to which the brain adapts in response to training and sensory interaction with the environment.

The listing or discussion of a prior-published document or any background in this specification should not necessarily be taken as an acknowledgement that the document or background is part of the state of the art or is common general knowledge. One or more aspects/embodiments of the present disclosure may or may not address one or more of the background issues.

### SUMMARY

According to a first aspect, there is provided an apparatus comprising:
at least one processor; and
at least one memory including computer program code, the at least one memory and computer program code configured to, with the at least one processor, cause the apparatus to:
receive a sensor output from a mechanomyography sensor configured to monitor muscle activity of a muscle in a human or animal body; and
control, in response to the received sensor output, an electrical stimulus applied by a muscle stimulator to the muscle to modify said muscle activity, wherein the electrical stimulus is applied with an amplitude below the motor threshold of the muscle simultaneously during monitoring of the muscle activity by the mechanomyography sensor.

The term “muscle” may be taken to encompass one or more muscles (e.g. a single muscle or a muscle group) and the associated sensory nerves linked to the muscle (directly or indirectly).

The muscle activity may be involuntary, and the apparatus may be configured to control the electrical stimulus to decrease the involuntary muscle activity.

The electrical stimulus and sensor output may each comprise a periodic or pseudo-periodic signal, and the apparatus may be configured to control the phase of the periodic or pseudo-periodic signal of the electrical stimulus relative to that of the sensor output to decrease the involuntary muscle activity.

The periodic or pseudo-periodic signal of the sensor output may comprise a higher frequency component within a lower frequency envelope, and the apparatus may be configured to control the phase of the periodic or pseudo-periodic signal of the electrical stimulus relative to that of the lower frequency envelope of the sensor output to decrease the involuntary muscle activity.

The apparatus may be configured to control the electrical stimulus such that the periodic or pseudo-periodic signal of the electrical stimulus has a phase difference of substantially ±180° relative to the lower frequency envelope.

The apparatus may be configured to control the electrical stimulus such that the periodic or pseudo-periodic signal of the electrical stimulus has an amplitude which is proportional to that of the lower frequency envelope.

The apparatus may be configured to compare the amplitude of the lower frequency envelope to a first predefined threshold defining an actionable level of involuntary muscle activity, and cause application of the electrical stimulus only if the amplitude of the lower frequency envelope exceeds the first predefined threshold.

A first mechanomyography sensor and muscle stimulator may be associated with an agonist muscle of an agonist/antagonistic pair and a second mechanomyography sensor and muscle stimulator may be associated with an antagonist muscle of the agonist/antagonistic pair. The apparatus may be configured to control the electrical stimulus applied by the second muscle stimulator to the antagonist muscle such that the periodic or pseudo-periodic signal of the electrical stimulus is substantially in-phase with the lower frequency envelope of the sensor output received from the first mechanomyography sensor associated with the agonist muscle, and vice-versa.

The apparatus may be configured to control the electrical stimulus applied by the second muscle stimulator to the antagonist muscle such that the periodic or pseudo-periodic signal of the electrical stimulus has a phase difference of substantially 0°, 330-30° or 90-270° relative to the lower frequency envelope of the sensor output received form the first mechanomyography sensor associated with the agonist muscle, and vice-versa.

The apparatus may be configured to:
receive the sensor output from each mechanomyography sensor during a predefined time period;
determine the lower frequency envelope of the sensor output during the predefined time period; and
predict the lower frequency envelope of the sensor output during a subsequent predefined time period for use in controlling the electrical stimulus during the subsequent predefined time period.

The apparatus may be configured to:
receive the sensor output from each mechanomyography sensor during the subsequent predefined time period;
determine the lower frequency envelope of the sensor output during the subsequent predefined time period;
determine a prediction error between the predicted and determined lower frequency envelopes of the sensor output during the subsequent predefined time period; and
predict, by accounting for the prediction error, the lower frequency envelope of the sensor output during a next subsequent predefined time period for use in controlling the electrical stimulus during the next subsequent predefined time period.

Each of the predefined, subsequent predefined and next subsequent predefined time periods may have substantially the same length.

The apparatus may be configured to filter the sensor output to increase a signal contribution from the involuntary muscle activity.

The muscle activity may be voluntary, and the apparatus may be configured to control the electrical stimulus to increase the voluntary muscle activity.

The apparatus may be configured to cause application of the electrical stimulus immediately upon receipt of the sensor output.

A first mechanomyography sensor and muscle stimulator may be associated with an agonist muscle of an agonist/antagonistic, pair and a second mechanomyography sensor and muscle stimulator may be associated with an antagonist muscle of the agonist/antagonistic pair. The apparatus may be configured to cause application of the electrical stimulus by the first muscle stimulator to the agonist muscle immediately upon receipt of the sensor output from the first mechanomyography sensor, and cause application of the electrical stimulus by the second muscle stimulator to the antagonist muscle immediately upon receipt of the sensor output from the second mechanomyography sensor.

The apparatus may be configured to filter the sensor output to increase a signal contribution from the voluntary muscle activity.

The apparatus may be configured to compare the sensor output to a second predefined threshold defining an actionable level of voluntary muscle activity, and cause application of the electrical stimulus only if an amplitude of the sensor output exceeds the second predefined threshold.

The second predefined threshold may be defined according to a noise baseline of the mechanomyography sensor.

The sensor output from the first mechanomyography sensor associated with the agonist muscle may be received simultaneously with the sensor output from the second mechanomyography sensor associated with the antagonist muscle.

The electrical stimulus may be applied as one or more stimulation bursts, and the apparatus may be configured to correlate the sensor output with the one or more stimulation bursts to identify induced muscle activity as a result of the applied stimulation.

The apparatus may be configured to decrease an amplitude of the electrical stimulus if the induced muscle activity exceeds a third predefined threshold defining an actionable level of induced muscle activity.

The apparatus may be configured to determine the third predefined threshold by increasing the amplitude of the electrical stimulus until the sensor output indicates that the muscle has contracted.

The apparatus may be configured to receive a further sensor output from an inertial measurement unit configured to monitor movement of the human or animal body, and control the electrical stimulus in response to the received further sensor output.

The apparatus may be configured to control at least one parameter of the electrical stimulus in response to one or more of the sensor output and further sensor output.

The apparatus may be configured to process one or more of the sensor output and further sensor output using a classifier to determine a severity of a neuromuscular disorder, and control at least one parameter of the electrical stimulus in response to the determined severity.

The apparatus may comprise one or more of the mechanomyography sensor and the muscle stimulator.

The mechanomyography sensor may comprise one or more of an acoustic sensor, an accelerometer, a piezoelectric sensor and a force sensor.

The muscle stimulator may comprise one or more electrode pairs configured to apply an electrical current to stimulate the muscle.

The one or more electrode pairs may be configured for transcutaneous or percutaneous electrical stimulation of the muscle.

According to a second aspect, there is provided a method comprising:
receiving a sensor output from a mechanomyography sensor configured to monitor muscle activity of a muscle in a human or animal body; and
controlling, in response to the received sensor output, an electrical stimulus applied by a muscle stimulator to the muscle to modify said muscle activity, wherein the electrical stimulus is applied with an amplitude below the motor threshold of the muscle simultaneously during monitoring of the muscle activity by the mechanomyography sensor.

According to a third aspect, there is provided an apparatus comprising:
at least one processor; and
at least one memory including computer program code, the at least one memory and computer program code configured to, with the at least one processor, cause the apparatus to:
receive a sensor output from at least one sensor configured to monitor muscle activity and/or movement of a human or animal body; and
control, in response to the received sensor output, an electrical stimulus applied by a muscle stimulator to a muscle in the body to modify said muscle activity and/or movement.

The at least one sensor may comprise one or more of an electromyography sensor, a mechanomyography sensor and an inertial measurement unit.

The electrical stimulus may be applied with an amplitude above or below the motor threshold of the muscle.

The electrical stimulus may be applied simultaneously during monitoring of the muscle activity and/or movement by the at least one sensor. On the other hand, the sensing and stimulation may be performed alternately.

According to a fourth aspect, there is provided a method comprising:
receiving a sensor output from at least one sensor configured to monitor muscle activity and/or movement of a human or animal body; and
controlling, in response to the received sensor output, an electrical stimulus applied by a muscle stimulator to a muscle in the body to modify said muscle activity and/or movement.

According to a fifth aspect, there is provided an apparatus as substantially described herein with reference to, and as illustrated by, the accompanying drawings.

The optional features described in relation to the apparatus of the first aspect are also applicable to the apparatus of the third and fifth aspects where compatible.

The steps of any method disclosed herein do not have to be performed in the exact order disclosed, unless explicitly stated or understood by the skilled person.

Corresponding computer programs (which may or may not be recorded on a carrier) for implementing one or more of the methods disclosed herein are also within the present disclosure and encompassed by one or more of the described example embodiments.

The present disclosure includes one or more corresponding aspects, example embodiments or features in isolation or in various combinations whether or not specifically stated (including claimed) in that combination or in isolation. Corresponding means for performing one or more of the discussed functions are also within the present disclosure.

The above summary is intended to be merely exemplary and non-limiting.

### BRIEF DESCRIPTION OF THE FIGURES

A description is now given, by way of example only, with reference to the accompanying schematic drawings, in which:—

FIG. 1 shows a direct muscle stimulation and monitoring method (schematic);

FIG. 2 shows an indirect muscle stimulation and monitoring method (schematic);

FIG. 3 shows one example of the present apparatus (schematic);

FIG. 4 shows one example an MMG sensor (cross-section);

FIG. 5a shows one example of an MMG sensor output for a stroke patient (graphical representation);

FIG. 5b shows an associated electrical stimulus for the MMG sensor output of FIG. 5a (graphical representation);

FIG. 6a shows one example of an MMG sensor output for an agonist muscle of a tremor patient (graphical representation);

FIG. 6b shows one example of an electrical stimulus for the agonist muscle of FIG. 6a (graphical representation);

FIG. 6c shows one example of an MMG sensor output for an antagonist muscle of a tremor patient (graphical representation);

FIG. 6d shows one example of an electrical stimulus for the antagonist muscle of FIG. 6c (graphical representation);

FIG. 7 shows a method of using the present apparatus (flow-chart);

FIG. 8 shows a computer-readable medium comprising a computer program configured to perform, control or enable the method of FIG. 7 (schematic)

FIG. 9 shows another method of using the present apparatus (flow-chart);

FIG. 10a shows the application of the present apparatus to an agonist/antagonist pair of muscles of a tremor patient (schematic);

FIG. 10b shows the application of the present apparatus to an agonist/antagonist pair of muscles of a stroke patient (schematic);

FIG. 11 shows a possible signal processing flow within the MMG sensor (schematic);

FIG. 12 shows the profiles of an MMG signal and associated electrical stimulus relative to an involuntary threshold (graphical representation); and

FIG. 13 shows the profiles of an MMG signal and associated electrical stimulus relative to involuntary and induced predefined thresholds (graphical representation),

### DESCRIPTION OF SPECIFIC ASPECTS/EMBODIMENTS

Physiological symptoms of neuromuscular disorders can include unwanted involuntary muscle activity (e.g. from essential tremor or Parkinson's) and weakened voluntary muscle activity (e.g. from Stroke or spinal cord injury). These symptoms can, however, be improved by monitoring the muscle activity of a patient and providing electrical stimulation to the affected muscle or muscle group.

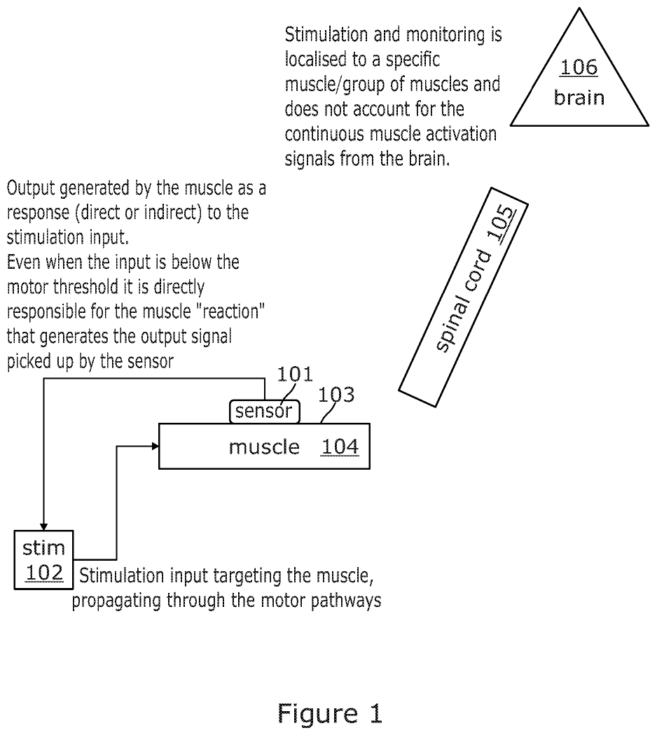

FIG. 1 shows how this can be achieved using one or more sensors 101 and a muscle stimulator 102. As illustrated, the one or more sensors 101 are attached to the patient for detecting movement or motor intent. For example, electromyography (EMG) or mechanomyography (MMG) sensors 101 may be placed on the surface of the patient's skin 103 in proximity to the affected muscle 104 to monitor muscle activity. Additionally or alternatively, an inertial measurement unit (not shown) may be attached to part of the patient's body (e.g. an arm or leg) to monitor movement of that body part. A muscle stimulator 102 is also attached to the patient to provide electrical stimulation to the affected muscle 104 based on the output of the sensors 101. The muscle stimulator 102 may comprise one or more electrode pairs each configured to apply an electrical current to stimulate the muscle 104. The electrode pairs could be surface electrodes placed on the surface of the patient's skin 103 and configured for transcutaneous electrical stimulation of the muscle 104, or they could be intramuscular electrodes inserted through the patient's skin 103 for percutaneous electrical stimulation.

Once the sensors 101 and muscle stimulator 102 are in place, the patient is asked to perform a known diagnostic movement which allows the sensors 101 to monitor the movement and/or muscle activity so that the severity of the neuromuscular disorder can be assessed. In some cases, the sensor output may be processed using a classifier to characterise the (intended) movement. The muscle stimulator 102 is then used to apply an electrical stimulus to the muscle 104 to modify the muscle activity. As will be described in more detail later, the form of the electrical stimulus can be tailored to the sensor output to treat the specific symptoms of the patient. In this respect, the classifier may be used to determine one or more parameters of the electrical stimulus in response to the characterised movement. For involuntary muscle activity such as tremors, the electrical stimulation signal may be provided destructively with the sensor output to weaken/inhibit or even cancel the unwanted movement. For voluntary muscle activity, on the other hand, the electrical stimulation signal may be provided constructively with the sensor output to strengthen the intended movement.

The movement and/or muscle activity of the patient continue to be monitored simultaneously during application of the electrical stimulus, thus providing a primary feedback loop to detect any change in the patient's symptoms. The output from the sensors 101 is then used to adapt the electrical stimulus to enable further improvement of the symptoms.

An important parameter of the applied stimulation signal is the amplitude. In the example of FIG. 1, the electrical stimulus is applied with an amplitude above the motor threshold of the muscle 104. As a result, the stimulation signal targets the efferent neurons and propagates through the motor pathways of the central nervous system. In this way, the stimulation signal at least partially blocks the impaired muscle activation signals sent down the spinal cord 105 by the brain 106 thereby attempting to address the problem locally. In this scenario, therefore, the muscle 104 is stimulated by the stimulation signal alone or in combination with the impaired signals from the brain 106 (i.e. the muscle activity is modified directly by the electrical stimulus). An issue with this approach, however, is that the symptoms of the neuromuscular disorder tend to appear again momentarily once the electrical stimulation is stopped.

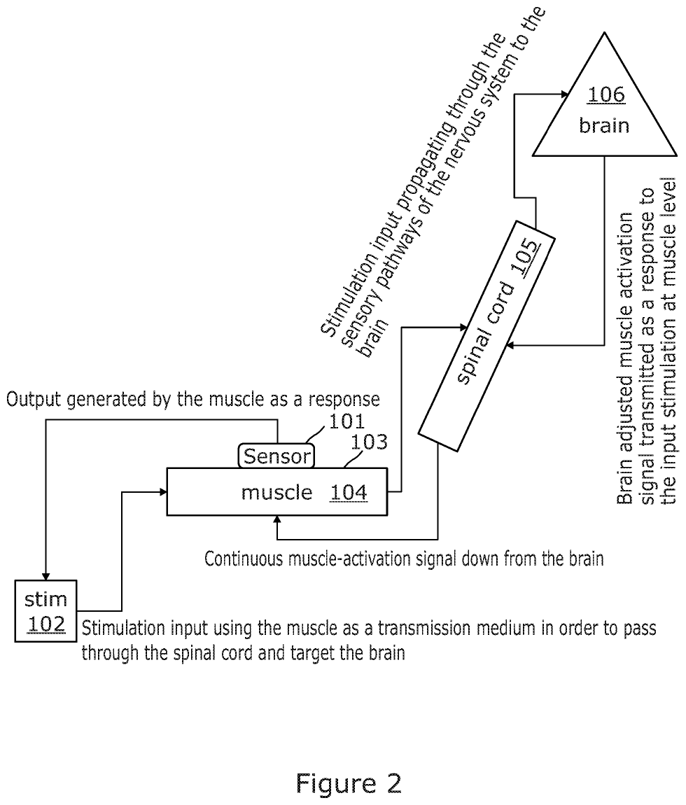

FIG. 2 shows an alternative approach that can result in a longer lasting effect. The components of the system are the same as those shown in FIG. 1, but this time the electrical stimulus is applied with an amplitude below the motor threshold of the muscle 104. By limiting the amplitude in this way, the applied signal targets the afferent neurons and propagates through the sensory pathways via the spinal cord 105 to the brain 106. As such, the signal does not interfere with the impaired muscle activation signals in the motor pathways. Rather, it stimulates the brain 106 and conditions it over time via brain plasticity. This has been found to correct the muscle activation signals generated by the brain 106 and thus reduce the effects of the neuromuscular disorder. In this scenario, therefore, the muscle 104 is stimulated by the natural (corrected) muscle activation signals transmitted by the brain 106 via the motor pathways (i.e. the muscle activity is modified indirectly by the electrical stimulus). Advantageously, these effects have been found to persist after removal of the applied stimulus. This approach therefore provides a therapeutic benefit instead of treating the presentation of the symptoms.

As with the previous example, the movement and/or muscle activity of the patient continue to be monitored simultaneously during application of the electrical stimulus and are used to adapt the parameters of the applied signal over time. Since the sensors 101 detect movement and/or muscle activity resulting from the corrected muscle activation signals from the brain 106, the sensor output provides a quantitative measurement of the therapeutic effect.

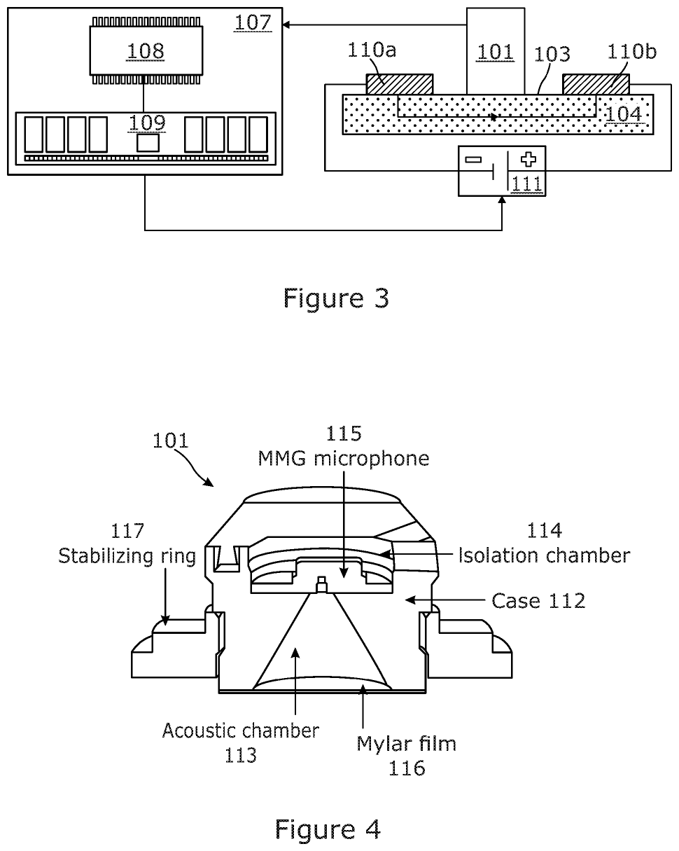

FIG. 3 shows one example of an apparatus 107 that may be used to perform the methods described above. The apparatus 107 comprises at least one processor 108 and at least one memory 109 including computer program code. The at least one memory 109 and computer program code are configured to, with the at least one processor 108, cause the apparatus 107 to receive a sensor output from at least one sensor 101 configured to monitor muscle activity and/or movement of a human or animal body and, in response to the received sensor output, control an electrical stimulus applied by a muscle stimulator 102 to a muscle 104 in the body to modify said muscle activity and/or movement.

One or both of the sensor 101 and muscle stimulator 102 may or may not form part of the apparatus 107. The sensor 101 may be an MMG sensor such as an acoustic sensor, an accelerometer, a piezoelectric sensor or a force sensor. An advantage of using an MMG sensor instead of an EMG sensor is that the measured signal is mechanical rather than electrical. Since the applied stimulus is electrical, the use of a mechanical sensor avoids the need for multiplexing two different types of electrical signals which could otherwise interfere with one another. Furthermore, when the stimulation and sensing are performed simultaneously, the stimulation signal (which may have a larger amplitude than the sensor signal) can drown out the sensor signal. The use of an MMG sensor therefore enables weaker muscle activity to be detected.

The muscle stimulator 102 may comprise one or more electrode pairs configured for transcutaneous or percutaneous electrical stimulation of the muscle 104. In this example, the muscle stimulator 102 comprises first 110a and second 110b surface electrodes attachable to the patient's skin 103. When a potential difference is applied between the first 110a and second 110b electrodes by a power supply 111, electrical current flows from the first electrode 110a through the underlying muscle 104 to the second electrode 110b. Although a single electrode pair is shown here, multiple electrode pairs could be used to increase the flow of current through the muscle 104. This may be useful for stimulating larger muscles, a group of muscles, or muscles with a relatively high activation threshold.

The processor 109 may be configured for general operation of the apparatus 107 by providing signalling to, and receiving signalling from, the other components to manage their operation. The storage medium 109 may be configured to store computer code configured to perform, control or enable operation of the apparatus 107. The storage medium 109 may also be configured to store settings for the other components. The processor 108 may access the storage medium 109 to retrieve the component settings in order to manage the operation of the other components. For example, the storage medium 109 may store the received sensor output together with corresponding (e.g. calibrated) settings for the muscle stimulator 102, and the processor 108 may utilise these settings to control the electrical stimulus applied by the muscle stimulator 102. The storage medium 109 may also store the first (“involuntary”), second (“voluntary”) or third (“induced”) predefined thresholds described later.

The processor 108 may be a microprocessor, including an Application Specific Integrated Circuit (ASIC). The storage medium 109 may be a temporary storage medium such as a volatile random access memory. On the other hand, the storage medium 109 may be a permanent storage medium such as a hard disk drive, a flash memory, or a non-volatile random access memory. The apparatus 107 may also comprise a power supply 111 (e.g. comprising one or more of a mains supply, a primary battery and a secondary battery) configured to provide each of the components with electrical power to enable their functionality.

Although not shown, the apparatus 107 may further comprise an electronic display (e.g. an LED, LCD or plasma display) configured to visually present the sensor output and/or electrical stimulus to a user of the apparatus 107, a loudspeaker configured to aurally present the sensor output and/or electrical stimulus to a user of the apparatus 107 and/or a transmitter configured to transmit the sensor output and/or electrical stimulus to a remote apparatus. The first (“involuntary”), second (“voluntary”) or third (“induced”) predefined thresholds may also be presented or transmitted together with the sensor output.

FIG. 4 shows one example of an acoustic MMG sensor that may be used to monitor the muscle activity. The sensor 101 comprises four components: a case 112 used to hold all of the parts together which defines an acoustic chamber 113 and an isolation chamber 114, a microphone 115 used to capture the muscle activity, a portion of transparent mylar film 116 used to amplify changes in pressure within the acoustic chamber 113, and a stabilizing ring 117 the dimensions of which have been determined to provide a snap-fit around the frontal side of the case 112. The stabilizing ring 117 causes the mylar film 116 to remain firmly stretched at the same time as preventing the case 112 from shifting or tilting. As the mylar film 116 is excited by a propagating muscle vibration, changes in air pressure within the acoustic chamber 113 are captured by the microphone 115. The microphone 115 itself is positioned on the backside of the case 112 at the bottom of the isolation chamber 114 which is sealed and filled with glue.

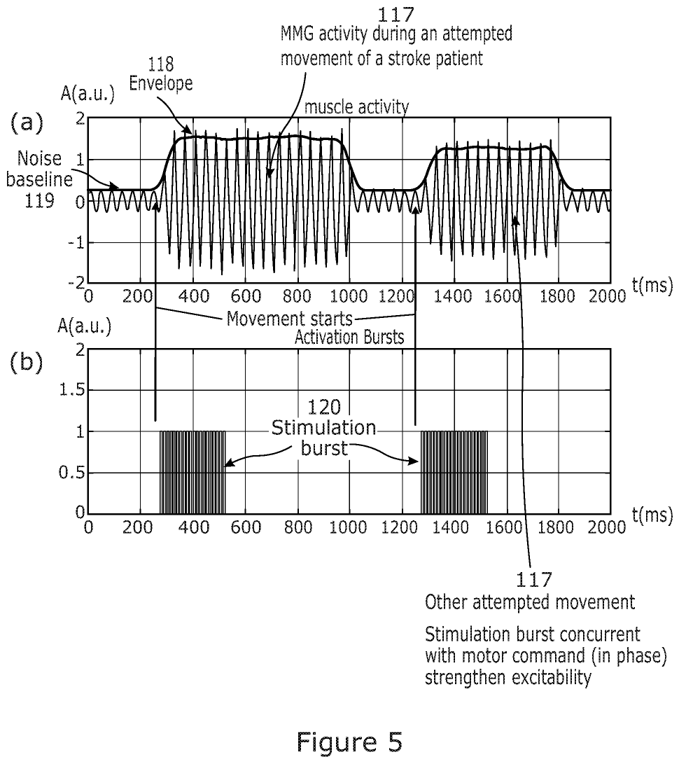

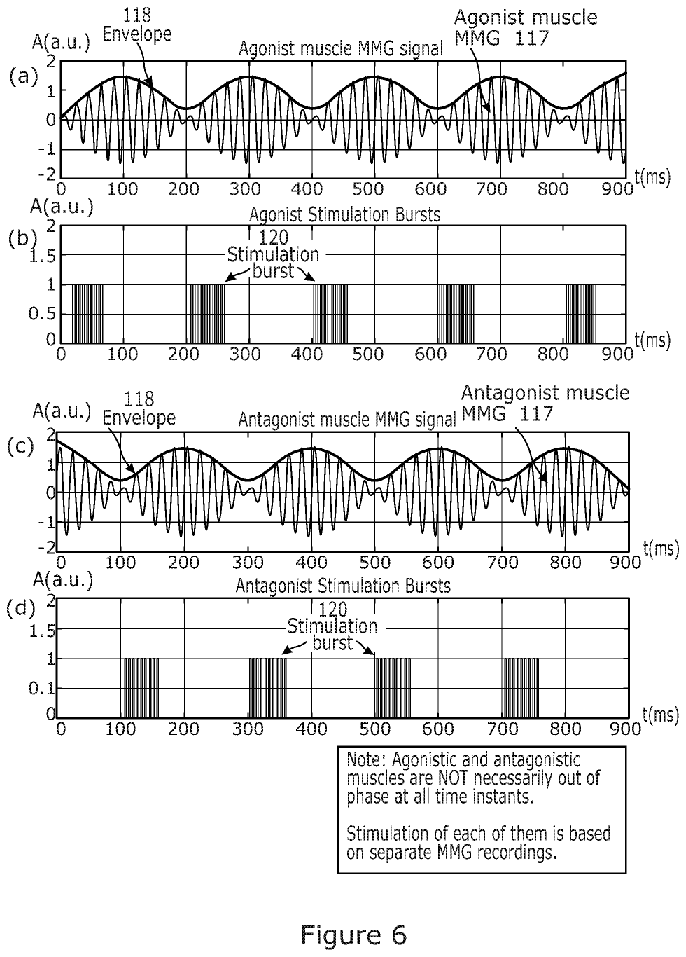

Application of the above-mentioned apparatus 107 and associated methods to stroke and tremor patients will now be described with reference to the signal waveforms shown in FIGS. 5 and 6, respectively. In these examples, one or more MMG sensors are used to monitor the muscle activity, and one or more electrode pairs are used to pass an electrical current through the associated muscle(s) with an amplitude below the motor threshold.

### Stroke Therapy (Voluntary Muscle Activity)

In this example, an MMG sensor and electrode pair are attached to a limb of a patient, and the patient follows a cue from a clinician (e.g. physiotherapist) or computer to attempt a predefined movement. Even when the patient is unable to complete the predefined movement, the MMG sensor is sufficiently sensitive to detect acoustic/mechanical waves caused by the muscle contractions.

FIG. 5a shows an example MMG sensor output for a stroke patient. The sensor output is plotted on a graph in which the x-axis denotes time in milliseconds and the y-axis denotes amplitude in arbitrary units. As shown, the signal comprises a substantially periodic (i.e. periodic or pseudo-periodic) component 117 associated with oscillations of the muscle fibres and a substantially uniform component 118 associated with the attempted movement. The substantially uniform component 118 modulates the amplitude of the substantially periodic component 117 and may therefore be referred to as the “envelope” of the signal. The envelope 118 is representative of the voluntary muscle activity and may be determined using an envelope detector implemented in hardware or software. To increase the signal contribution from the voluntary muscle activity, the apparatus may be configured to band-pass filter the sensor output, e.g, to exclude any signal components with a frequency outside of a 2-50 Hz range. Furthermore, the apparatus may be configured to compare the sensor output to a predefined “voluntary” threshold defining an actionable level of muscle activity and ignore/remove any signal components with an amplitude below this threshold as background noise. The predefined voluntary threshold may be defined according to a noise baseline 119 of the MMG sensor (e.g. 5 times the standard deviation of the noise baseline 119) and can be estimated between each movement attempt on a patient-specific basis. When the sensor output exceeds the predefined voluntary threshold, the apparatus detects the voluntary muscle activity which triggers the electrical stimulus.

FIG. 5b shows an example of an electrical stimulus applied to the muscle (i.e. the same muscle or muscle group being monitored by the sensor) in response to the sensor output of FIG. 5a. As with the sensor output, the electrical stimulus is plotted on a graph in which the x-axis denotes time in milliseconds and the y-axis denotes amplitude in arbitrary units. The electrical stimulus is applied as a single stimulation burst 120 for each detected movement attempt. For example, the stimulation burst 120 may have an amplitude of less than 6 mA, a burst frequency of around 100 Hz and a duration of up to 500 ms. The timing and amplitude of the electrical stimulus are important. For stroke therapy, the electrical stimulus should ideally be applied immediately upon detection of the voluntary muscle activity, but preferably no later than 50 ms from said detection in order to induce associative brain plasticity. In addition, the amplitude of the electrical stimulus should be kept below the motor threshold of the muscle such that it targets the afferent neurons for “indirect” stimulation. To help ensure this, the apparatus may be configured to correlate the sensor output with the stimulation bursts 120 to provide a secondary feedback loop for identifying induced muscle activity as a result of the applied stimulation. In this context, the term “induced muscle activity” implies that the electrical stimulus has exceeded the motor threshold and is targeting the efferent neurons (i.e. “direct” muscle stimulation). If the induced muscle activity exceeds a predefined “induced” threshold defining an actionable level of induced muscle activity, the apparatus may be configured to decrease the amplitude of the electrical stimulus until the induced muscle activity is below the predefined induced threshold. The predefined induced threshold may be determined by increasing the amplitude of the electrical stimulus until the sensor output indicates that the muscle has contracted. Preferably, the amplitude of the electrical stimulus should be set as high as possible without inducing a detectable muscle response.

The patient is typically asked to repeat the (attempted) movement multiple times during the therapy session with a period of rest between consecutive attempts. The rest period may vary from one patient to the next but should be sufficient to avoid pain or muscle fatigue. A suitable rest period might be 5-10 seconds. Each time voluntary muscle activity is detected, a stimulation burst 120 is applied to the muscle. This establishes an associative effect between the command sent by the patient's brain and the sensory feedback provided by the electrical stimulation.

### Tremor Treatment (Involuntary Muscle Activity)

In this example, an MMG sensor and electrode pair are positioned adjacent each muscle of an agonist/antagonist pair (e.g. biceps and triceps in the upper arm or wrist flexors and extensors in the forearm), and the MMG sensors monitor involuntary muscle activity of the agonist and antagonist muscles independently.

FIGS. 6a and 6c show an example MMG sensor output for the agonist and antagonist muscles, respectively. As shown, the sensor output is a substantially periodic (i.e. periodic or pseudo-periodic) signal comprising a higher frequency component 117 associated with oscillations of the muscle fibres and a lower frequency (“envelope”) component 118 associated with the tremor. The envelope 118 is representative of the involuntary muscle activity and may be determined using an envelope detector implemented in hardware or software. The apparatus may be configured to band-pass filter the sensor output to increase the signal contribution from the involuntary muscle activity relative to any voluntary muscle activity not affected by tremor, e.g. to exclude any signal components with a frequency outside of a 4-10 Hz range. Furthermore, the apparatus may be configured to compare the amplitude of the envelope 118 to a predefined “involuntary” threshold defining an actionable level of involuntary muscle activity and ignore/remove any signal components with an amplitude below this threshold as background noise. When the amplitude of the envelope 118 exceeds the predefined involuntary threshold, the apparatus tracks the phases of the agonist and antagonist envelopes 118 substantially in real-time for use by the muscle stimulator. This could be performed using a phase-locked loop or via the Hilbert transform, for example.

FIGS. 6b and 6d show examples of respective electrical stimuli applied to the agonist and antagonist muscles in response to the sensor outputs of FIGS. 6a and 6c. In order to suppress the tremor, each electrical stimulus comprises a series of stimulation bursts 120 which together form a substantially periodic (periodic or pseudo-periodic) signal. The electrical stimulus applied to the agonist muscle (FIG. 6b) is substantially out-of-phase with the envelope 118 of the sensor output from the agonist muscle shown in FIG. 6a and is substantially in-phase with the envelope 118 of the sensor output from the antagonist muscle shown in FIG. 6c. Similarly, the electrical stimulus applied to the antagonist muscle (FIG. 6d) is substantially out-of-phase with the envelope 118 of the sensor output from the antagonist muscle shown in FIG. 6c and is substantially in-phase with the envelope 118 of the sensor output from the agonist muscle shown in FIG. 6a. The expression “substantially out-of-phase” may be taken to mean that the electrical stimulus has a phase difference of 90-270°, 150-210° or substantially 180° relative to the envelope 118 of the sensor output from the muscle being stimulated, and the expression “substantially in-phase” may be taken to mean that the electrical stimulus has a phase difference of substantially 0°, 330-30° or 90-270° relative to the envelope 118 of the sensor output from the other muscle of the agonist/antagonist pair. The stimulation is applied below the motor threshold of the agonist and antagonist muscles but may have an amplitude which is proportional to (and possibly even matches) that of the envelope 118. In some cases, the stimulation bursts 120 may have an amplitude of less than 6 mA and a burst frequency of around 100 Hz. As with the stroke therapy, the apparatus may be configured to monitor the direct effect of the electrical stimulation on the sensor output in order to keep any induced muscle activity below the predefined “induced” threshold. The predefined induced threshold may be determined by increasing the amplitude of the electrical stimulus until the sensor output indicates that the muscle has contracted.

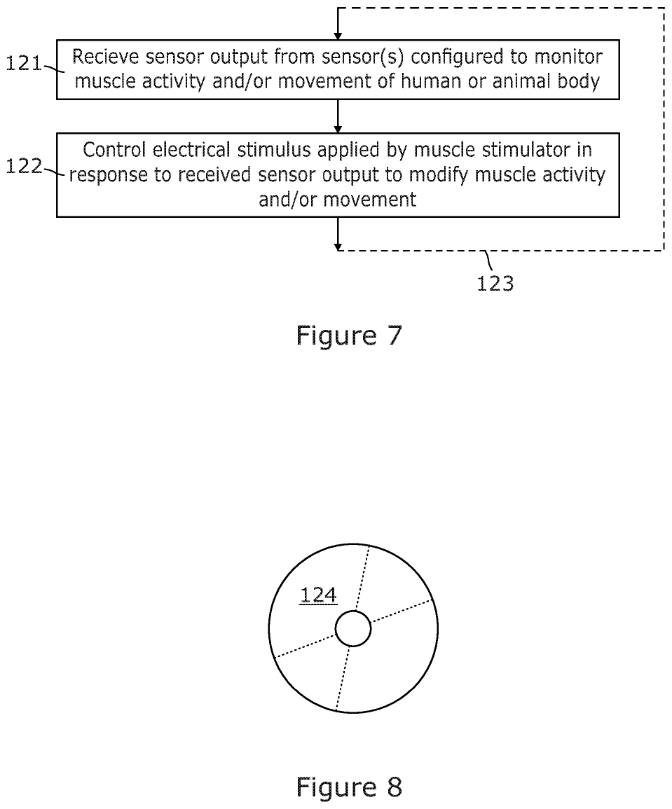

FIG. 7 shows schematically the main steps 121-122 of a method of using the present apparatus. The method generally comprises: receiving a sensor output from at least one sensor configured to monitor muscle activity and/or movement of a human or animal body 121; and controlling, in response to the received sensor output, an electrical stimulus applied by a muscle stimulator to the muscle to modify said muscle activity and/or movement 122. Furthermore, the muscle activity may be monitored simultaneously during application of the electrical stimulus as indicated by the primary feedback loop 123.

FIG. 8 illustrates schematically a computer/processor readable medium 124 providing a computer program according to one example. The computer program may comprise computer code configured to perform, control or enable one or more of the method steps 121-122 of FIG. 7 using an apparatus 107 described herein. In this example, the computer/processor readable medium 124 is a disc such as a digital versatile disc (DVD) or a compact disc (CD). In other embodiments, the computer/processor readable medium 124 may be any medium that has been programmed in such a way as to carry out an inventive function. The computer/processor readable medium 124 may be a removable memory device such as a memory stick or memory card (SD, mini SD, micro SD or nano SD).

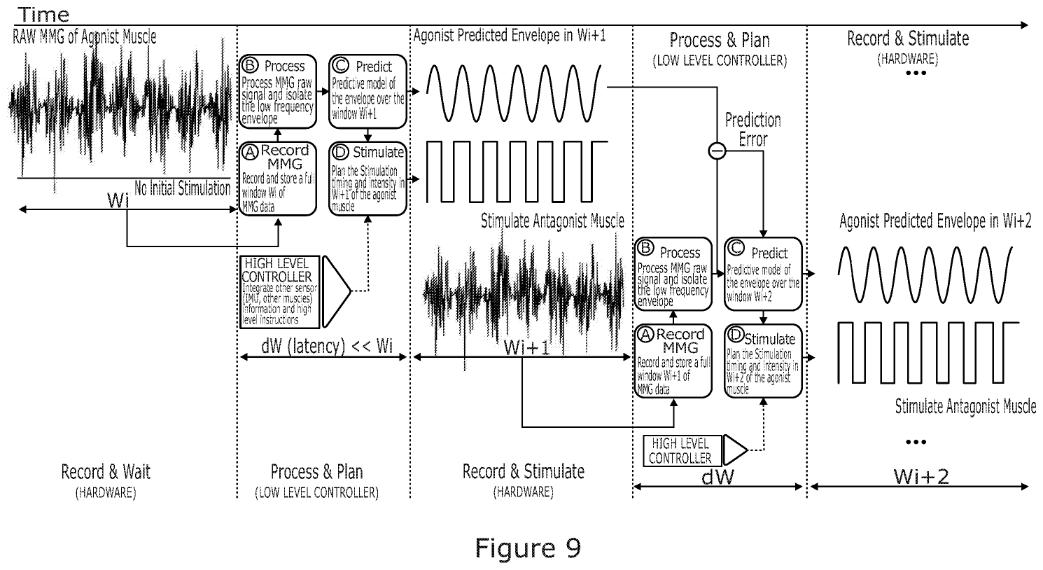

FIG. 9 shows a flow chart for processing a received MMG signal and controlling the associated electrical stimulus to decrease involuntary muscle activity. In this example, the MMG signal has been received from an agonist muscle of a tremor patient. Depending on how much MMG signal data needs to be processed, and the amount of time required to perform this processing, it may be necessary to predict the future envelope of the sensor output in order to apply a suitable electrical stimulus to decrease the involuntary muscle activity.

As shown in step A, a recording buffer is used to store the raw MMG signal collected during consecutive predefined time periods (or recording windows Wᵢ). In this example, the recording windows have the same length (although they could vary slightly) and there is no overlap between two consecutive recording windows. In step B, a processor then analyses the raw MMG signal during an interval dW between consecutive recording windows to determine the lower frequency envelope.

The processing interval dW typically depends on the length of the recording window W but should be substantially shorter than Wᵢ to minimise latency and ensure stability. Also, depending on the processing and control strategy, longer or shorter recording windows may be preferable. Longer recording windows provide a greater volume of data from which to analyse the MMG signal. However, longer windows also rely on a more accurate prediction of the future envelope in the consecutive window Wᵢ₊₁ than shorter windows and make the system more intermittent.

After processing the raw MMG signal from the agonist muscle to determine the lower frequency envelope for window Wᵢ, the processor predicts the lower frequency envelope for the consecutive window Wᵢ₊₁ in step C. This is then used to determine the electrical stimulus uᵢ₊₁ to be applied during window Wᵢ₊₁. As will be described in more detail later, the electrical stimulus in step D is applied in-phase to an antagonist muscle of the tremor patient.

Simultaneously while the muscle stimulator is applying the electrical stimulus, the recording buffer stores new MMG data received from the MMG sensor during window Wᵢ₊₁. The process is then repeated for the next consecutive window Wᵢ₊₂. However, because both recorded and predicted lower frequency envelopes are available for window Wᵢ₊₁, any error in the previous prediction can be accounted for when predicting the lower frequency envelope for the next consecutive window Wᵢ₊₂. This improves the effectiveness of the subsequent stimulation. Information from one or more other sensors (e.g. an inertial measurement unit) can also be taken into account to further improve the effectiveness of the stimulation.

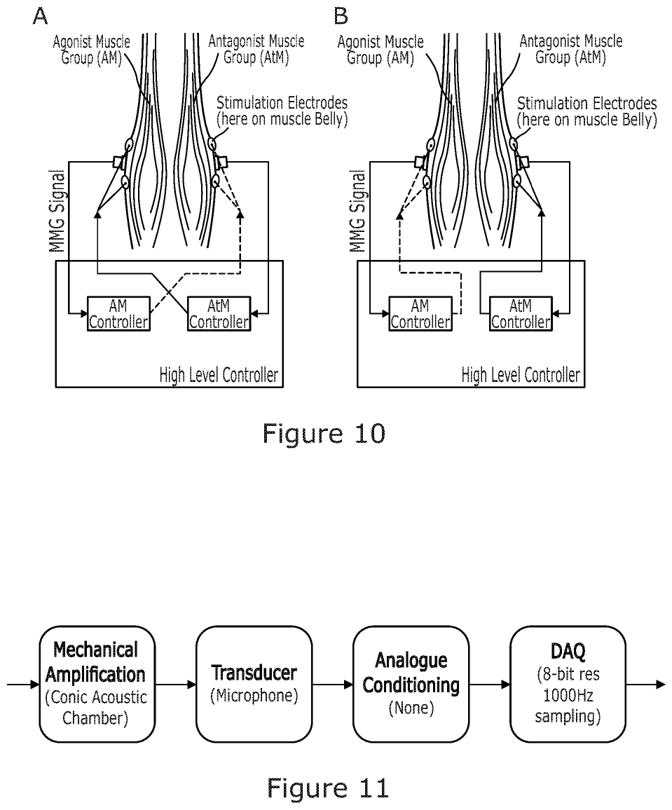

FIGS. 10a and 10b show the application of the present apparatus to an agonist/antagonist pair of muscles of a tremor patient and stroke patient, respectively. In this example, the electrical stimulus is applied to the belly of the muscle, but it could be applied to other parts of the muscle instead (including the associated sensory nerves linked to the muscle for indirect stimulation). A first MMG sensor and muscle stimulator are associated with the agonist muscle of the agonist/antagonist pair and a second MMG sensor and muscle stimulator are associated with the antagonist muscle of the agonist/antagonist pair. The sensor output from the first MMG sensor associated with the agonist muscle may be received simultaneously with the sensor output from the second MMG sensor associated with the antagonist muscle. Furthermore, each muscle stimulator comprises a pair of electrodes configured to enable a flow of electrical current through the muscle.

As shown in FIG. 10a, an agonist muscle (AM) controller is configured to control the electrical stimulus applied by the second muscle stimulator to the antagonist muscle such that the periodic or pseudo-periodic signal of the electrical stimulus is below the motor threshold and substantially in-phase with the lower frequency envelope of the sensor output received from the first MMG sensor associated with the agonist muscle. Similarly, an antagonist muscle (AtM) controller is configured to control the electrical stimulus applied by the first muscle stimulator to the agonist muscle such that the periodic or pseudo-periodic signal of the electrical stimulus is below the motor threshold and substantially in-phase with the lower frequency envelope of the sensor output received from the second MMG sensor associated with the antagonist muscle. In some cases, the electrical stimulus may have a phase difference of substantially 0°, 330-30° or 90-270° relative to the lower frequency envelope of the sensor output. This “cross-stimulation” approach has been found to activate a proprioceptive/cutaneous spinal reflex that naturally dampens the tremor activity without inducing any counter-acting muscular contraction. A higher-level controller may also be used to coordinate the AM and AtM controllers and integrate other sensor information as needed.

As shown in FIG. 10b, there is no “cross-stimulation” of the agonist/antagonist pair when treating the stroke patient. Rather, the AM controller is configured to cause application of the electrical stimulus by the first muscle stimulator to the agonist muscle below the motor threshold and immediately upon receipt of the sensor output from the first MMG sensor. Similarly, the AtM controller is configured to cause application of the electrical stimulus by the second muscle stimulator to the antagonist muscle below the motor threshold and immediately upon receipt of the sensor output from the second MMG sensor.

FIG. 11 shows an example of a signal processing flow within an MMG sensor such as the one illustrated in FIG. 4. In this example, the noise or pressure waves emanating from the muscular contraction are amplified by the acoustic chamber. The amplified pressure waves are then transduced into an analogue signal by the microphone mounted at the back of the chamber. Analogue conditioning (filtering or amplification) could at this stage be applied to the analogue signal but is not essential or performed in the current design. Finally, a digital acquisition system (DAQ) converts the analogue signal into a digital signal.

The digital processing of the signal could involve separating activity associated with voluntary movement from that associated with involuntary movement, Amplification of smaller higher frequency components could also be implemented. The estimation period or pseudo-period of the involuntary movement signal could help narrow isolation of the lower frequency envelope. The lower frequency envelope of the MMG signals may require de-modulation from the higher frequency components in a specific frequency range. The MMG signals may also be prone to different types of artefacts resulting from voluntary or involuntary motion which could require removal.

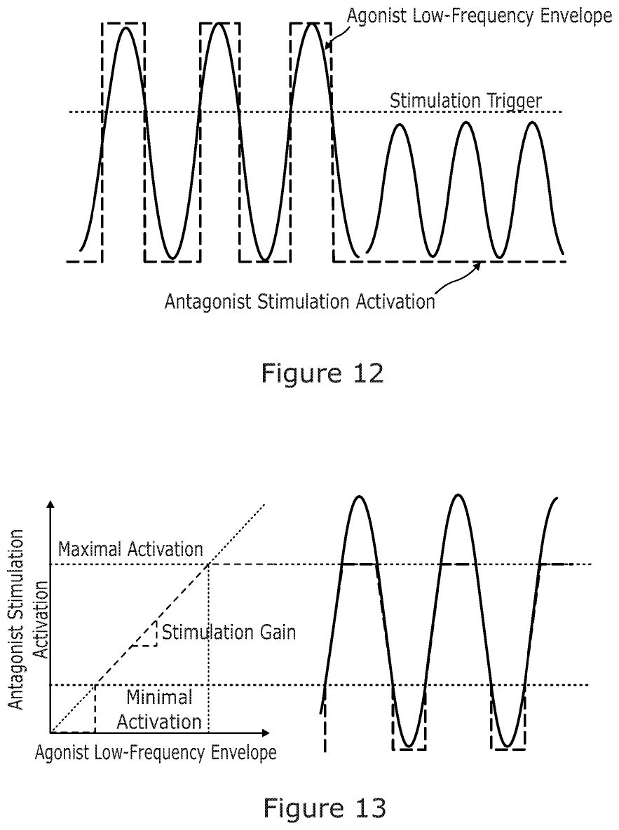

FIG. 12 shows the profiles of an MMG signal and associated electrical stimulus relative to a first predefined (involuntary) threshold defining an actionable level of involuntary muscle activity. As illustrated, the resulting antagonist electrical stimulation trajectory is completely in-phase with respect to the agonist lower frequency envelope.

FIG. 13 shows an antagonist electrical stimulation trajectory proportional to an agonist lower frequency envelope. A first (involuntary) threshold defining an actionable level of involuntary muscle activity is used to withhold the stimulation in the absence of sufficient involuntary movement, and a third (induced) threshold defining an actionable level of induced muscle activity is used to saturate the stimulation output below motor-threshold.

The applicant hereby discloses in isolation each individual feature described herein and any combination of two or more such features, to the extent that such features or combinations are capable of being carried out based on the present specification as a whole, in the light of the common general knowledge of a person skilled in the art, irrespective of whether such features or combinations of features solve any problems disclosed herein, and without limitation to the scope of the claims. The applicant indicates that the disclosed aspects/embodiments may consist of any such individual feature or combination of features. In view of the foregoing description it will be evident to a person skilled in the art that various modifications may be made within the scope of the disclosure.

1. An apparatus comprising:
at least one processor; and
at least one memory including computer program code, the at least one memory and computer program code configured to, with the at least one processor, cause the apparatus to:
receive a sensor output from a mechanomyography sensor configured to monitor muscle activity of a muscle in a human or animal body; and
control, in response to the received sensor output, an electrical stimulus applied by a muscle stimulator to the muscle to modify said muscle activity, wherein the electrical stimulus is applied with an amplitude below the motor threshold of the muscle simultaneously during monitoring of the muscle activity by the mechanomyography sensor.

2. The apparatus of claim 1, wherein the muscle activity is involuntary, and the apparatus is configured to control the electrical stimulus to decrease the involuntary muscle activity.

3. The apparatus of claim 2, wherein the electrical stimulus and sensor output each comprise a periodic or pseudo-periodic signal, and wherein the apparatus is configured to control the phase of the periodic or pseudo-periodic signal of the electrical stimulus relative to that of the sensor output to decrease the involuntary muscle activity.

4. The apparatus of claim 3, wherein the periodic or pseudo-periodic signal of the sensor output comprises a higher frequency component within a lower frequency envelope, and wherein the apparatus is configured to control the phase of the periodic or pseudo-periodic signal of the electrical stimulus relative to that of the lower frequency envelope of the sensor output to decrease the involuntary muscle activity.

5. The apparatus of claim 4, wherein the apparatus is configured to control the electrical stimulus such that the periodic or pseudo-periodic signal of the electrical stimulus has an amplitude which is proportional to that of the lower frequency envelope.

6. The apparatus of claim 5, wherein the apparatus is configured to compare the amplitude of the lower frequency envelope to a first predefined threshold defining an actionable level of involuntary muscle activity, and cause application of the electrical stimulus only if the amplitude of the lower frequency envelope exceeds the first predefined threshold.

7. The apparatus of claim 2, wherein a first mechanomyography sensor and muscle stimulator are associated with an agonist muscle of an agonist/antagonistic pair and a second mechanomyography sensor and muscle stimulator are associated with an antagonist muscle of the agonist/antagonistic pair, and wherein the apparatus is configured to control the electrical stimulus applied by the second muscle stimulator to the antagonist muscle such that the periodic or pseudo-periodic signal of the electrical stimulus is substantially in-phase with the lower frequency envelope of the sensor output received from the first mechanomyography sensor associated with the agonist muscle, and vice-versa.

8. The apparatus of claim 7, wherein the apparatus is configured to control the electrical stimulus applied by the second muscle stimulator to the antagonist muscle such that the periodic or pseudo-periodic signal of the electrical stimulus has a phase difference of substantially 0°, 330-30° or 90-270° relative to the lower frequency envelope of the sensor output received form the first mechanomyography sensor associated with the agonist muscle, and vice-versa.

9. The apparatus of claim 7, wherein the apparatus is configured to:
receive the sensor output from each mechanomyography sensor during a predefined time period;
determine the lower frequency envelope of the sensor output during the predefined time period; and
predict the lower frequency envelope of the sensor output during a subsequent predefined time period for use in controlling the electrical stimulus during the subsequent predefined time period.

10. The apparatus of claim 9, wherein the apparatus is configured to:
receive the sensor output from each mechanomyography sensor during the subsequent predefined time period;
determine the lower frequency envelope of the sensor output during the subsequent predefined time period;
determine a prediction error between the predicted and determined lower frequency envelopes of the sensor output during the subsequent predefined time period; and
predict, by accounting for the prediction error, the lower frequency envelope of the sensor output during a next subsequent predefined time period for use in controlling the electrical stimulus during the next subsequent predefined time period.

11. The apparatus of claim 10, wherein each of the predefined, subsequent predefined and next subsequent predefined time periods have substantially the same length.

12. The apparatus of claim 2, wherein apparatus is configured to filter the sensor output to increase a signal contribution from the involuntary muscle activity.

13. The apparatus of claim 1, wherein the muscle activity is voluntary, and the apparatus is configured to control the electrical stimulus to increase the voluntary muscle activity.

14. The apparatus of claim 13, wherein the apparatus is configured to cause application of the electrical stimulus immediately upon receipt of the sensor output.

15. The apparatus of claim 13, wherein a first mechanomyography sensor and muscle stimulator are associated with an agonist muscle of an agonist/antagonistic pair and a second mechanomyography sensor and muscle stimulator are associated with an antagonist muscle of the agonist/antagonistic pair, and wherein the apparatus is configured to cause application of the electrical stimulus by the first muscle stimulator to the agonist muscle immediately upon receipt of the sensor output from the first mechanomyography sensor, and cause application of the electrical stimulus by the second muscle stimulator to the antagonist muscle immediately upon receipt of the sensor output from the second mechanomyography sensor.

16. The apparatus of claim 13, wherein the apparatus is configured to filter the sensor output to increase a signal contribution from the voluntary muscle activity.

17. The apparatus of claim 13, wherein the apparatus is configured to compare the sensor output to a second predefined threshold defining an actionable level of voluntary muscle activity, and cause application of the electrical stimulus only if an amplitude of the sensor output exceeds the second predefined threshold.

18. The apparatus of claim 17, wherein the second predefined threshold is defined according to a noise baseline of the mechanomyography sensor.

19. The apparatus of claim 7, wherein the sensor output from the first mechanomyography sensor associated with the agonist muscle is received simultaneously with the sensor output from the second mechanomyography sensor associated with the antagonist muscle.

20. The apparatus of claim 1, wherein the electrical stimulus is applied as one or more stimulation bursts, and wherein the apparatus is configured to correlate the sensor output with the one or more stimulation bursts to identify induced muscle activity as a result of the applied stimulation.

21. The apparatus of claim 20, wherein the apparatus is configured to decrease an amplitude of the electrical stimulus if the induced muscle activity exceeds a third predefined threshold defining an actionable level of induced muscle activity.

22. The apparatus of claim 21, wherein the apparatus is configured to determine the third predefined threshold by increasing the amplitude of the electrical stimulus until the sensor output indicates that the muscle has contracted.

23. The apparatus of claim 1, wherein the apparatus is configured to receive a further sensor output from an inertial measurement unit configured to monitor movement of the human or animal body, and control the electrical stimulus in response to the received further sensor output.

24. The apparatus of claim 23, wherein the apparatus is configured to control at least one parameter of the electrical stimulus in response to one or more of the sensor output and further sensor output.

25. The apparatus of claim 23, wherein the apparatus is configured to process one or more of the sensor output and further sensor output using a classifier to determine a severity of a neuromuscular disorder, and control at least one parameter of the electrical stimulus in response to the determined severity.

26. The apparatus of claim 1, wherein the apparatus comprises one or more of the mechanomyography sensor and the muscle stimulator.

27. The apparatus of claim 26, wherein the mechanomyography sensor comprises one or more of an acoustic sensor, an accelerometer, a piezoelectric sensor and a force sensor.

28. The apparatus of claim 26, wherein the muscle stimulator comprises one or more electrode pairs configured to apply an electrical current to stimulate the muscle.

29. The apparatus of claim 28, wherein the one or more electrode pairs are configured for transcutaneous or percutaneous electrical stimulation of the muscle.

30. A method comprising:
receiving a sensor output from a mechanomyography sensor configured to monitor muscle activity of a muscle in a human or animal body; and
controlling, in response to the received sensor output, an electrical stimulus applied by a muscle stimulator to the muscle to modify said muscle activity, wherein the electrical stimulus is applied with an amplitude below the motor threshold of the muscle simultaneously during monitoring of the muscle activity by the mechanomyography sensor.

31. A computer program comprising computer code configured to perform the method of claim 30.
